(19) United States
(12) Patent Application Publication
(10) Pub. No.: US 2011/0226258 A1
(43) Pub. Date: Sep. 22, 2011

(54) Uterus Implant

(76) Inventor: Suzan Gail Black, Dayton, TX (US)

(21) Appl. No.: 12/648,370

(22) Filed: Mar. 19, 2010

(51) Int. Cl. A61F 6/06

(52) U.S. Cl. 128/834

(57) ABSTRACT

A uterine implant device suitable for treatment of vaginal vault prolapsed which occurs in a women after a partial or total hysterectomy. The implant device is formed in the shape of a normal uterus and filled with a suitable fluid. The uterine implant device is placed within the pelvic vault and can be held in place by sutured attached ligaments or scar tissue. The implant device can be inserted utilizing surgery or a specialized insertion tool.

### TECHNICAL FIELD

The present invention relates to an implant and a method for the treatment of the vaginal vault prolapse for women who have had a hysterectomy

### BACKGROUND

Prolapse is a relatively common condition, particularly amongst older women. It occurs when the pelvic floor muscles become weak or damaged and can no longer support the pelvic organs. These organs are then able to push against the walls of the vagina causing women to feel discomfort and “something coming down”. Uterine prolapse occurs when the womb drops down into the vagina and vaginal vault prolapse can occur in women who have had a hysterectomy.

Vaginal vault prolapse is when the walls of the vagina fall in on themselves. This can happen after the uterus has been removed. Vaginal vault prolapse occurs when the upper portion of the vagina loses its normal shape and sags or drops down into the vaginal canal or outside of the vagina. It may occur alone or along with prolapse of the bladder, urethra, rectum or small bowel. Vaginal vault prolapse is usually caused by weakness of the pelvic and vaginal tissues and muscles. Normally, surgery is needed to repair the problem and relieve the symptoms. Prolapse of the posterior vaginal wall has been treated by surgically attaching a mesh to the posterior portion of the vagina.

Breast implants utilizing silicone gel technology is known in the prior art. Women use the breast implants to cosmetically enlarge their breast or to replace their breasts after a mastectomy. There are two primary types of breast implants: saline-filled and silicone-gel-filled implants. Saline implants have a silicone elastomer shell filled with sterile saline liquid. Silicone gel implants have a silicone shell filled with a viscous silicone gel. The present invention relates to a novel uterine implant made from silicone gel technology for use by women who are experiencing vaginal vault prolapse.

### SUMMARY

One of the major objectives of the present invention is to provide an effective method of treating vault prolapsed in women who have had a hysterectomy.

This patent entails the use of breast implant silicone gel technology filled to create a uterus. This implant is designed to keep the bladder in the proper area of the pelvic cavity after total or partial hysterectomy. It takes the place of the uterus as safe as breast implants, preventing bladder from “falling” in the void. It is held in place with “attachment ligament sheath” using only a few sutures or scar tissue.

### BRIEF DESCRIPTION OF THE DRAWINGS

FIG. 1 illustrates a frontal view of the female reproductive system with a normal uterus.

FIG. 1a illustrates a side view of the female reproductive system with a normal uterus.

FIG. 2 illustrates an exploded view of the female reproductive system;

FIG. 2a is an exploded frontal view showing a partial hysterectomy.

FIG. 2b is an exploded frontal view showing a total hysterectomy.

FIG. 3 is side view showing the uterus removed.

FIG. 4 is a frontal perspective view of present invention, the uterine implant.

FIG. 5 is a bottom view of the uterine implant showing the valve;

FIG. 6 is a cross-sectional view of valve.

7 frontal view showing implant disposed on top of vaginal canal.

FIG. 7a is a side view showing implant within vault of uterus.

### DETAILED DESCRIPTION

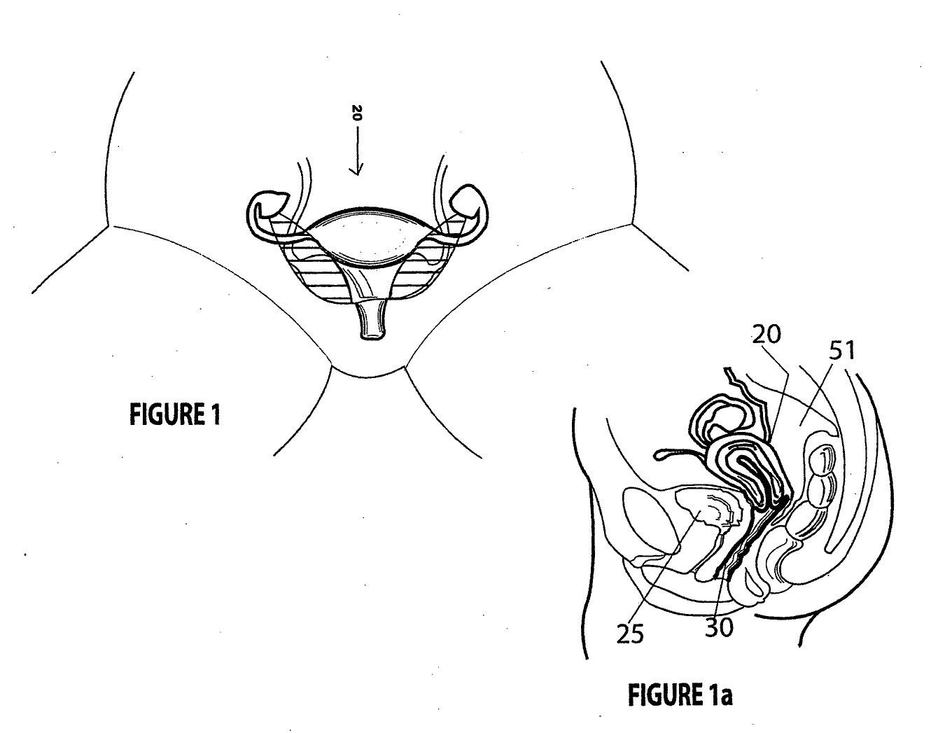

Referring to FIGS. 1 and 1a there is shown respectively a frontal view and side view of a normal female reproductive system prior to a hysterectomy. A normal female reproductive system comprises the uterus (20), cervix (21), fallopian tubes (22), and ovaries (23). The uterus (20) is a pear-shaped organ located between the bladder and the lower intestine. As depicted, the uterus (20) is positioned above the vaginal canal (30). A normal uterus (20) is comprised of muscles that expand during pregnancy and provide support for surrounding organs. When the woman is not pregnant, the uterus (20) is the size of a fist with the walls collapsed and flattened against each other. The cervix (21) is the lower portion of the uterus (20). It has a canal opening into the vagina called the os. The os allows the menstrual blood to flow out of the uterus into the vagina. The fallopian tubes are two tubes that are positioned on each side of the side. An ovary is positioned near the end of each tube. Ovaries (23) are the egg-producing organs of the female reproductive system.

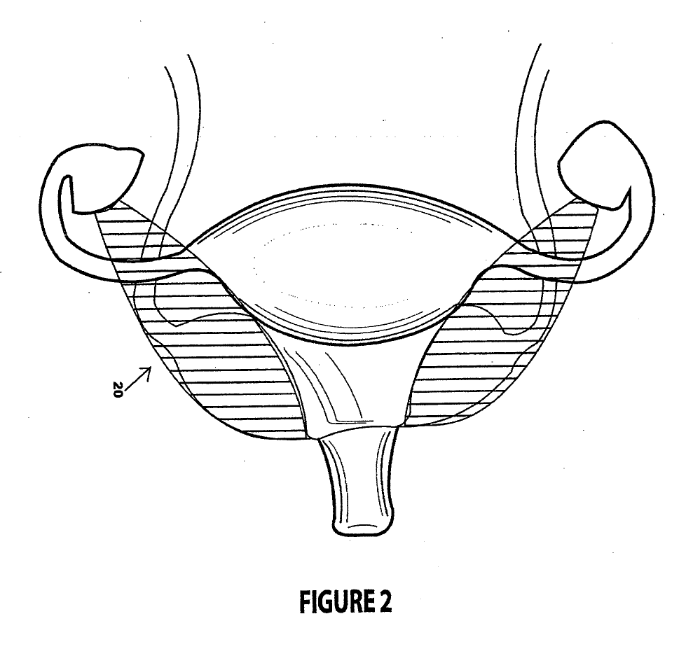

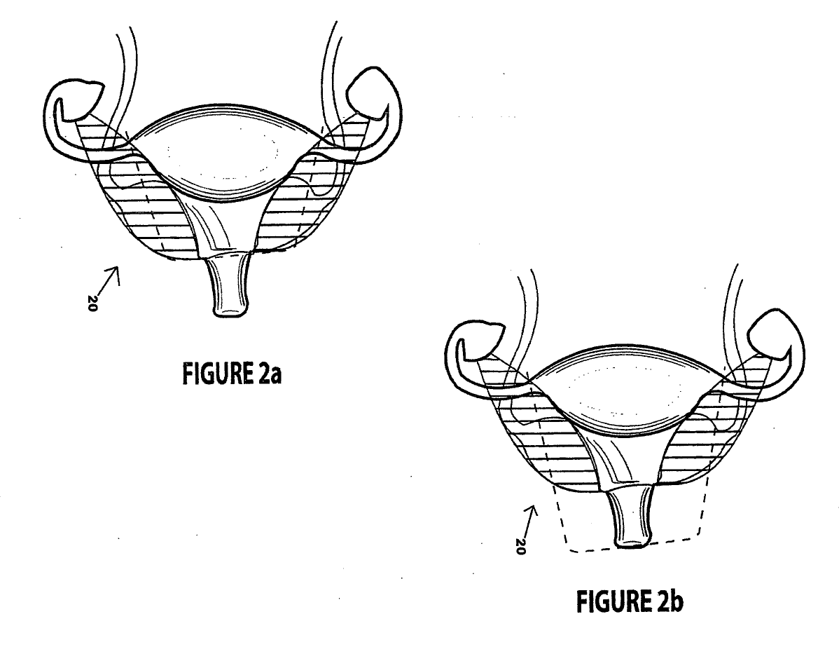

After a hysterectomy, women become susceptible to vault prolapse because the removal of the supporting uterine muscles creates a vault (51) within the pelvic cavity. A woman can have a partial hysterectomy as shown in FIG. 2a or a total hysterectomy as shown in FIG. 2b. A partial hysterectomy is just the removal of the uterus (20). A total hysterectomy is the removal of the uterus (20) and the cervix (21). Thus, the present invention is adapted to support both a partial and a total hysterectomy.

The normal uterus (20) has a broad ligament (24) that connects the sides of the uterus (20) to the walls and floor of the pelvis (not shown). The broad ligament (24) comprises (1) mesometrium which is the largest portion of the broad ligament; (2) mesosalpinx which surrounds the uterine tube; (3) mesovarium which connects the anterior surface of the ovary to the remainder of the broad ligament. The broad ligament is a flat sheath the encompasses the uterus and extends beyond the opposing sides of the uterus and it helps to maintain the uterus, ovaries and the uterine tubes in its position.

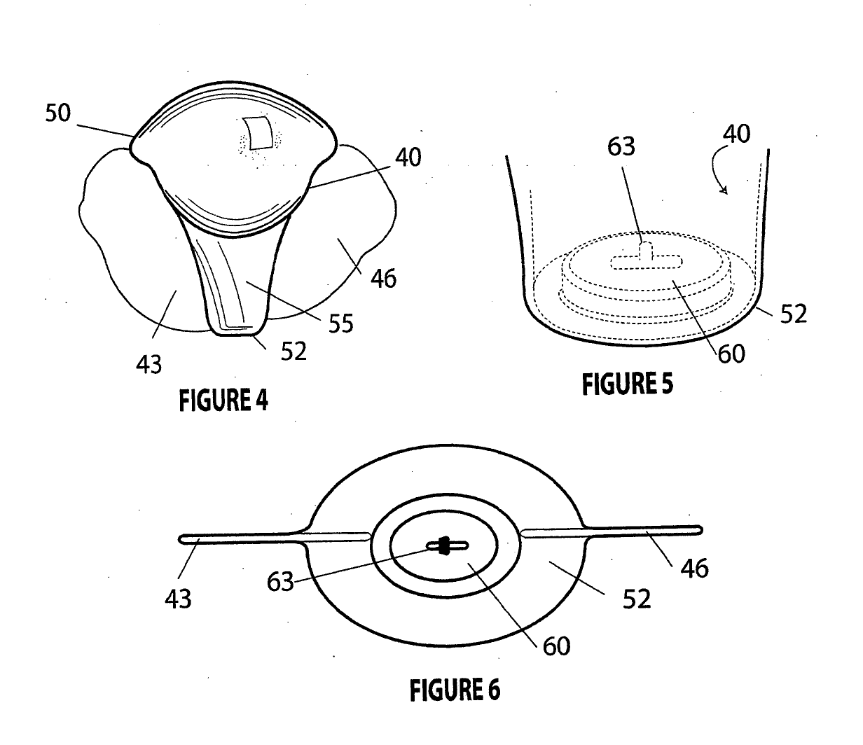

Referring to FIG. 4, the present invention comprises a collapsible sack member (40) formed in the shape of a normal female uterus which is generally pear shape and is dimensioned to the size of a normal female uterus. Sack member (40) has a surrounding external shell or wall (55) made of silicone with a cavity for receiving sterile filling fluid therein.

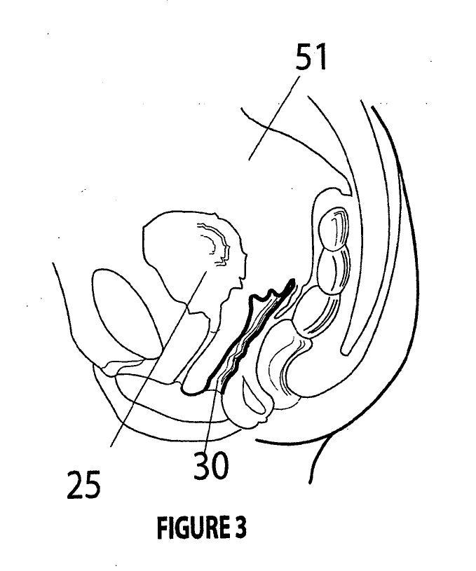

Referring to FIGS. 4, 5, and 6, sack member (40) is further defined by an upper end (50) and a lower end (52). Valve (60) is located at the lower end (52) of the sack member (40) and is adapted with a fluid communication port (63) for receiving fluid within the interior cavity of sack member (40). The outer shell (55) can be smooth or textured. A textured outer shell allows the body to form a seal. A sack container (40) with a hollow interior cavity formed in shape of a uterus as shown in FIG. 3. The surrounding outer wall (55) is made of a silicone material similar to the material used in breast implants. The valve (60) is disposed and coupled to the lower end of sack container (40). Ligaments (43, 46) are attached to the opposing upper ends of sack container (40) and extend laterally therefrom.

Referring to FIGS. 5 and 6, valve (60) is a conventional valve and is adapted with fluid communication port 63 to allow engagement with an insertion device (not shown) for fluid communication into the sack (40). The insertion device (not shown) would be adapted with a container of filling fluid which can be sterile saline water (salt water) or another suitable sterile filling fluid. The insertion tool can be a standard medical syringe which is filled with a fluid.

After the unfilled sack member (40) is inserted into the pelvic area, the insertion tool is inserted through the vaginal area to engage with the valve located at the lower end of sack member (40). Once inserted, orifice at the end of the conventional syringe is inserted into the valve and the liquid is pumped into the cavity of sack member (40).

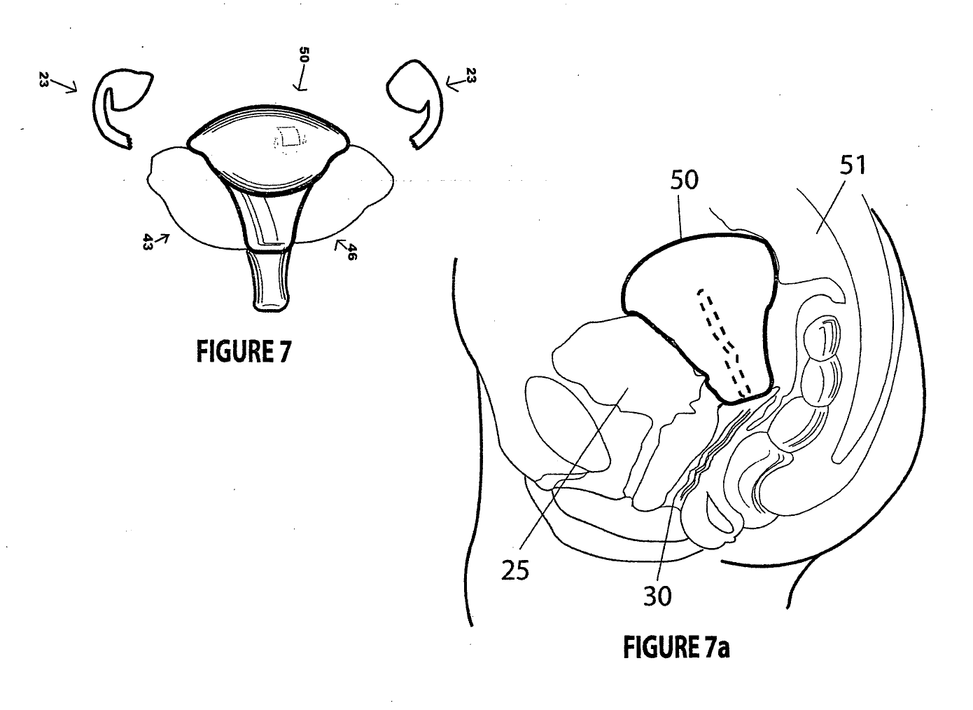

For insertion into the pelvic cavity, the empty sack (40) is folded and inserted into the vaginal vault (51). Then, the insertion tool can be inserted through the vagina and injected into the self-sealing valve member (63). Then ligaments (42, 43) can be extended outward and sutured in place to allow the sack member (40) to be suspended in its normal position thereby preventing vault prolapse as shown in FIGS. 7 and 7a.

In an alternative embodiment, sack member (40) can have a textured outer shell. Once the textured sack member (40) is inserted into the pelvic area, the body sees it as a foreign body and forms surrounding scar tissue thereby further assisting in maintaining the implant in place. After sack container (40) is inserted, the body will form scar tissue that adheres to the surrounding wall (55) thereby securing the uterus implant (40) within vault (51) in the pelvic area of a woman.

In use, the sack container (40) can be implanted surgically within the vault after a hysterectomy. Once inserted surgically, the sack member (40) can be filled with sterile fluid and then sutured in place within the pelvic cavity. Alternatively, the sack member as described above can be inserted utilizing a specialized insertion tool through the vaginal canal and into the pelvic vault.

What is claim is:

1. An uterine device designed to be implanted above the vaginal canal and inserted into a vault of a woman who has had a total or partial hysterectomy, the device comprising:
a sack member defined by an upper end and lower end;
the sack member surrounded by an outer external wall which is made of silicone gel type material with a cavity therein to receive a sterile filling fluid;
the sack member formed in the shape of a woman's normal size uterus, a valve disposed at a lower end of the sack member, the valve adapted to be in fluid communication with a specialized tool adapted for transferring the sterile filling fluid in the cavity of the sack member through the valve wherein the sack member becomes enlarged into the shape of a normal size uterus; and the sack member being implanted into the vault the woman and being sutured in place into the pelvic area.

2. The device of claim 1 where in the external wall is textured.

3. The device of claim 1 where the external wall is smooth.

4. The device of claim 1 wherein the external wall is made of silicone elastomer.

5. The device of claim 4 wherein the filling fluid is saline water.

6. The device of claim 1 wherein the filling fluid is viscous silicone gel.

7. The device of claim 1 further comprising:
a pair of ligaments being attached at opposing sides of the sack member and extending laterally outward therefrom;
the pair of ligament being made of silicone gel type of material that and each ligament having a peripheral end that is sutured in place within the vault of the woman wherein the sack member is held in place within the vault.

8. A method of insertion of an uterine implant device as defined in claim 7, the method comprising:
providing a flat and folded as implant device defined in claim 7;
providing an insertion tool having a container with sterile filling fluid;
inserting the implant device into the vault of a woman who have had a hysterectomy;
connecting the insertion tool to the valve located at the lower end of the uterine implant device causing the tool to be in fluid communication with cavity within the sack member; and
transferring the sterile fling fluid from the container of the insertion tool into the sack member through the valve located at the lower end of the sack member wherein the sack member becomes the size of a normal size uterus; and
suturing in place the pair of ligaments wherein the implant device is maintained in place within the vault.
